(19) United States
(12) Patent Application Publication
(10) Pub. No.: US 2006/0173366 A1
(43) Pub. Date: Aug. 3, 2006

(54) Vascular stiffness evaluation apparatus, vascular stiffness index calculating program, and vascular stiffness index calculating method

(76) Inventor: Motoharu Hasegawa, Tokyo (JP)

Correspondence Address: RADER FISHMAN & GRAUER PLLC, LION BUILDING, 1233 20TH STREET N.W., SUITE 501, WASHINGTON, DC, 20036, US

(21) Appl. No.: 11/344,106

(22) Filed: Feb. 1, 2006

(30) Foreign Application Priority Data
Feb. 2, 2005 (JP) 2005-026952

(51) Int. Cl. A61B 5/02

(52) U.S. Cl. 600/485; 600/500

(57) ABSTRACT

Investigation is made into a cause by which an abnormal result is obtained from an arterial stiffness evaluation apparatus for evaluating a degree of arteriosclerosis based on pulse waves and blood pressures which are detected from an ankle and an upper arm. Provided is an arterial stiffness evaluation apparatus capable of calculating an index of accurate vascular stiffness. A vascular stiffness evaluation apparatus (60) includes: a pulse wave detecting unit (10) including a strain sensor (13); a pulse wave velocity determining unit (30); a blood pressure detecting unit (10); and a vascular stiffness index calculating unit (50) for calculating a vascular stiffness index which serves as an index of vascular stiffness of a living body based on the pulse wave velocity and the blood pressures. The pulse wave detecting unit (10) includes a brachial pulse wave detecting unit (11) for detecting a brachial pulse wave of the living body and a popliteal pulse wave detecting unit (21) for detecting a popliteal pulse wave of the living body. The blood pressure detecting unit (40) includes a strain sensor (13).

### BACKGROUND OF THE INVENTION

1. Field of the Invention

The present invention relates to a vascular stiffness evaluation apparatus for evaluating vascular stiffness of a living body, particularly, arterial stiffness thereof, a vascular stiffness index calculating program for calculating and outputting a vascular stiffness index which serves as an index indicating vascular stiffness, and a vascular stiffness index calculating method of calculating the vascular stiffness index.

2. Description of the Related Art

The average length of life (life expectancy) for men and women in our country is increasing, while the birthrate therefor is decreasing every year, so an aging society is rapidly coming. Facing a change in society due to the aging of population, in order to avoid a decline of national economic power and a shortage or breakdown of a social security cost or a medical care cost, it is important that each and every individual in the country tries to promote his/her health, thereby preventing lifestyle-related diseases dramatically increasing.

A large number of lifestyle-related diseases which threaten the lives of the country's people are based on vascular diseases such as a stroke and a cardiac disease. Therefore, if the degree of arteriosclerosis leading to the vascular diseases can be simply and accurately measured, this is medically and socially useful.

Up to now, development have been made on various methods of diagnosing the arteriosclerosis. One of the methods is utilizing pulse wave propagation velocity (or “pulse wave velocity”, which is also referred to as “PWV”) of a living body (PWV method). A pulse wave indicates wave motion in the case where a change in internal pressure of a blood vessel which is caused when blood is forced to an aorta by the cardiac systole is propagated in a peripheral direction. The pulse wave velocity is velocity of the pulse wave propagating through the blood vessel. A healthy blood vessel is soft and has high elasticity, while a blood vessel in which the arteriosclerosis occurs is hard and easily broken, so the property of the pulse wave which quickly propagates through a hard material and slowly propagates through a soft material is utilized. Propagation velocity of the pulse wave propagated between two points in an artery is measured. When the propagation velocity becomes higher, it is diagnosed that stiffness of the blood vessel increases.

Pulse wave measurement examples known up to now will be described below. FIG. 12 shows a method which is called a Frank method. According to this method, two pulse wave sensors are used to detect a pulse wave of the carotid artery and a pulse wave of the femoral artery. Distances “a” and “b+c” between the aortic valve region and respective pulse wave detection points are measured. In view of a path in which the aorta extends, not a distance along a straight line but a distance along a broken line (sum of distances “b” and “c”) is measured between the aortic valve region and a pulse wave sensor for femoral artery detection.

The carotid artery pulse wave which is obtained by the carotid artery pulse wave sensor exhibits a waveform indicated by (a) as shown in FIG. 13. The femoral artery pulse wave which is obtained by the femoral artery pulse wave sensor exhibits a waveform indicated by (b) as shown in FIG. 13. Predetermined rising times of the pulse waves, for example, times, each of which corresponds to a time when a level value reaches 1/5 of a peak value are compared with each other to obtain a time difference therebetween as a time T. Therefore, when the distances “a”, “b” and “c” and the time T are obtained, the pulse wave velocity (PWV) is calculated by the following expression.
PWV=(b+c−a)/T Expression (3)

Next, FIGS. 14 and 15 shows a pulse wave velocity measuring method which is called a PWV original method. As shown in FIG. 14, according to this method, sensors are located in positions of the carotid artery and the femoral artery to detect pulse waves thereof. In addition, a heart sound sensor is located in the aortic valve region to detect a heart sound. A straight distance D between the aortic valve region and the femoral artery pulse wave sensor is measured. Then, the straight distance D is multiplied by 1.3 times to correct a difference with an actual arterial path.

Assume that a time difference between the rising of the carotid artery pulse wave shown in FIG. 15 and the rising of the femoral artery pulse wave is a time T. In addition, assume that a time between the production of an aortic valve closing sound of a second heart sound (forward component of second heart sound) and a carotid artery notch (inflection point of the carotid artery pulse wave) is a time tc. The pulse wave velocity (PWV) can be calculated by the following expression based on the above-mentioned values.
PWV=(D×1.3)/(t+tc) Expression (4)

The pulse wave velocity measured using the Frank method or the PWV original method varies depending on a blood pressure. This is because, when the blood pressure increases, a blood vessel expands, so that the blood vessel apparently becomes harder. Therefore, a large number of cases in which blood pressures are different from one another are statistically analyzed to determine a pulse wave velocity calibration curve as shown in FIG. 16. In the case of actual diagnosis of arteriosclerosis, a pulse wave velocity and a blood pressure are measured. The measured pulse wave velocity is converted into a pulse wave velocity measured when a minimum blood pressure is 80 mmHg based on the pulse wave velocity calibration curve. As a result, the pulse wave velocity is obtained without depending on the blood pressure. The diagnosis of arteriosclerosis is performed based on the pulse wave velocity obtained in view of the blood pressure condition. Therefore, when a blood pressure is measured at the time of pulse wave velocity measurement and a measured pulse wave velocity is calibrated based on the measured blood pressure, the calibrated pulse wave velocity can be useful for the diagnosis of the progress of arteriosclerosis or the diagnosis of various diseases caused by the arteriosclerosis. This is an arteriosclerosis diagnosing method based on the pulse wave velocity.

However, a state of arteriosclerosis to be evaluated based on the pulse wave velocity is significantly influenced by, for example, a blood pressure value or a personal difference, which is a factor other than an arteriosclerosis factor. Therefore, when a specific person is diagnosed at regular intervals, diagnosis results can be meaningfully used. On the other hand, when an unspecified person is diagnosed only one time, the influence of a personal difference is large, so that it is difficult to universally and objectively determine a diagnosis result.

In such a situation, a novel index capable of evaluating arterial stiffness based on the pulse wave velocity, a maximum blood pressure, and a minimum blood pressure has been developed by the inventors of the present invention. The detail is described in JP 2004-236730 A. the new index, that is, a vascular stiffness index (cardio ankle vascular index (CAVI)) is expressed by the following expression (5) or (6) based on the pulse wave velocity and a ratio between the maximum blood pressure and the minimum blood pressure.
CAVI₁=k₁·ln(Ps/Pd)·PWV² Expression (5)
CAVI₂=k₂·√[ln(Ps/Pd)]·PWV Expression (6)
(where k₁ and k₂ each denotes a constant)

In the case where the vascular stiffness index CAVI (each of CAVI₁ and CAVI₂ is referred to as CAVI) is used, when the pulse wave velocity, the maximum blood pressure, and the minimum blood pressure are measured, the vascular stiffness index is calculated based thereon. Therefore, there are advantages in that it is unnecessary to adjust a blood pressure value to a minimum blood pressure value, the maximum blood pressure and the minimum blood pressure which are obtained can be directly used, and the influence of the personal difference is small.

In an arterial stiffness evaluation apparatus for obtaining the vascular stiffness index CAVI (hereinafter referred to as a “CAVI apparatus”), upper arms and ankles are wound with cuffs. Then, the vascular stiffness index is calculated based on pulse waves and blood pressures which are obtained in such a state. Therefore, there are many advantages in that, for example, everyone can very simply perform the measurement, a burden on a person to be examined is small, an index reflecting a state of a lower-limb artery is obtained because the PWV of an artery including the lower-limb artery is measured. Thus, it is expected that the apparatus is widely used in a medical field.

The above-mentioned techniques and the apparatus for noninvasively measuring the arterial stiffness are disclosed in JP 61-119252 A, JP 01-259836 A, JP 03-015439 A, JP 05-038332 A, JP 2001-137203 A, JP 3140007 B, and JP 2004-236730 A.

However, it is found that, in some cases, a value which is unreasonable as the degree of arteriosclerosis is obtained even when the CAVI apparatus, which seems to be versatile, is used. The following data relate to 25 examples (the total number of measurement cases is 163 (the average number of measurements per case is 6 or more), age ranges from 24 to 81, 17 examples are the cases of men, and 8 examples are the cases of women) which are measured using the CAVI apparatus in three medical offices. Each of measurement data is converted from a CAVI value into a PWV value. The data are classified based on the followings.

First, for each of the examples, a stable low-value group is specified as a non-abnormal group with reference to a plurality of measurement values and an average value thereof is obtained. A high-value group in which the pulse wave velocity becomes 10% or more and less than 20% of the obtained average value is determined as an abnormal group “A” and a high-value group in which the pulse wave velocity becomes 20% or more of the obtained average value is determined as an abnormal group “B”. Results are shown in Tables 1 and 2.

TABLE 1
Ratio of Each Group to All Cases

| | Each Group/All Cases | Ratio (%) |
|---|---|---|
| Non-Abnormal Group | 129/163 | 79.14 |
| Abnormal Group A | 23/163 | 14.11 |
| Abnormal Group B | 11/163 | 6.75 |
| A + B | 34/163 | 20.86 |

TABLE 2
Average PWV Value of Each Group and Increase Degree thereof

| | Average | Each Case/Non- |
|---|---|---|
| | PWV Value | Abnormal Group |
| Non-Abnormal Group | 7.98 m/s ± 0.75 | 1 |
| Abnormal Group A | 9.96 m/s ± 0.36 | 1.13 |
| Abnormal Group B | 11.9 m/s ± 0.36 | 1.47 |
| A + B | 10.59 m/s ± 0.70 | 1.24 |

As is apparent from Table 1, of 163 current subject cases in total, approximately 14% is the abnormal group “A” and approximately 6.6% is the abnormal group “B”, that is, approximately 20.9% which is the sum of both is not normal. As is apparent from Table 2, an average of the PWV value of the non-abnormal group is 7.98±1.25, while an average PWV value of the abnormal group “A” is 9.96±1.79 and an average PWV value of the abnormal group “B” is 11.90±2.18. As a result, a PWV value ratio of the abnormal group “A” to the non-abnormal group is 1.3 times and a PWV value ratio of the abnormal group “B” to the non-abnormal group is 1.47 times.

When the study to find the cause of an abnormal value which does not reflect the degree of arteriosclerosis and the improvement for preventing the abnormal value are not performed, a person to be examined have a false perception of arterioscleros in some cases even when the CAVI apparatus which is simple and useful is used. Therefore, the disadvantage of society increases.

### SUMMARY OF THE INVENTION

Thus, an object of the present invention is to investigate a cause by which an abnormal value is included in a result obtained by measurement using the CAVI apparatus and to provide an improved CAVI apparatus capable of calculating a numeral value which is an index of accurate vascular stiffness.

That is, an object of the present invention is to provide a vascular stiffness evaluation apparatus capable of simply and accurately obtaining a vascular stiffness index on which a degree of arteriosclerosis of a living body is reflected, a program for calculating the vascular stiffness index, and a method of calculating the vascular stiffness index.

In order to achieve the above-mentioned objects, there is provided a vascular stiffness evaluation apparatus, including: pulse wave detecting means for detecting pulse waves, which includes a strain sensor; pulse wave velocity determining means for determining a pulse wave velocity based on the pulse waves; blood pressure detecting means for detecting blood pressures; and vascular stiffness index calculating means for calculating a vascular stiffness index which serves as an index of vascular stiffness of a living body based on the pulse wave velocity and the blood pressures.

A conventional arterial stiffness evaluation method using a pulse wave velocity is based on the long-standing accumulation of data and the result of research and has contributed to a medical care for a long time. On the other hand, the reason why an abnormal value is obtained in an arteriosclerosis diagnosis method (hereinafter referred to as a “CAVI” method) using the CAVI apparatus, which is an improved PWV method, is studied. As a result, because a state of arteriosclerosis of a lower limb artery can be evaluated using the CAVI method, it is determined that not only an aorta but also limb arteries are included as measurement subjects. That is, in the case of the CAVI method, pulse wave velocities of blood vessels of various regions of the body can be measured and pulse wave measurement is performed on the limb arteries. However, when a region lower than a knee for a leg and a region anterior to an elbow for an arm are stimulated, vasoreflex or vasospasm occurs, so that there is a problem in that the pulse wave velocity significantly increases. The vasoreflex and the vasospasm is a phenomenon in which stiffness of a vascular wall increases without any change of a vascular diameter and a phenomenon in which the stiffness of the vascular wall increases while the vascular diameter reduces, respectively. In peripheral limb arteries, those phenomenons are likely to cause by cold, mental stress, compression, or the like. The vasoreflex or the vasospasm may be caused by a stimulus of the cuff wound around the ankle or the wrist.

In the present invention, the pulse wave detecting means including the strain sensor is used. The strain sensor includes the semiconductor strain gauge provided on a metal plate such as a stainless steel plate or a phosphorus bronze plate. Although the strain sensor has a simple structure, there is an advantage in which the pulse wave of a measurement region can be directly detected thereby. A pressure applied to the living body to detect the pulse wave is the order of 10 mmHg, so that it is substantially unnecessary to compress the living body. Therefore, the vasoreflex or the vasospasm can be suppressed. In addition, mental and physical burdens on a person to be examined can be reduced. According to the present invention, a vascular stiffness evaluation apparatus includes pulse wave detecting means including a strain sensor, pulse wave velocity determining means, blood pressure detecting means, and vascular stiffness index calculating means for calculating a vascular stiffness index which serves as an index of vascular stiffness of a living body based on a pulse wave velocity and blood pressures, so the vascular stiffness index is calculated based on the pulse wave velocity and the blood pressures. Therefore, when there are data related to the pulse wave velocity and the blood pressures, the vascular stiffness can be speedily and accurately evaluated without being affected by a variation in blood pressure and using, for example, calibration data set in advance.

It is possible to use a vascular stiffness evaluation apparatus in which the pulse wave detecting means includes brachial pulse wave detecting means for detecting a brachial pulse wave of the living body and popliteal pulse wave detecting means for detecting a popliteal pulse wave of the living body. Because the pulse wave detecting means includes the brachial pulse wave detecting means for detecting the brachial pulse wave of the living body and the popliteal pulse wave detecting means for detecting the popliteal pulse wave of the living body, it is unnecessary to detect a pulse wave of the region lower than the knee for the leg and a pulse wave of the region anterior to the elbow for the arm. Therefore, an accurate vascular stiffness index can be obtained without being affected by the vasoreflex or the vasospasm. The pulse wave is detected from each of a brachial region and a popliteal region, so the pulse wave can be simply and accurately measured by everyone. In addition, the mental and physical burdens on the person to be examined are little. The pulse wave is detected from each of the brachial region and the popliteal region, so that the vascular stiffness index can be calculated without performing pulse wave detection and heart sound detection on other regions.

The vascular stiffness index can be based on a value (stiffness) obtained by the following expression (1)
stiffness=ln(Ps/Pd)×k·PWV² Expression (1)
(where Ps denotes a maximum blood pressure, Pd denotes a minimum blood pressure, PWV denotes the pulse wave velocity, and k denotes a constant).

A predetermined blood vessel has a relationship in which a squared pulse wave velocity (PWV²) increases as a logarithm pulse pressure (ln(Ps/Pd)) decreases. The product of the logarithm pulse pressure and the squared pulse wave velocity is a value inherent to a subject blood vessel. Therefore, because the stiffness value obtained by the above-mentioned expression (1) is used for the vascular stiffness index, a factor resulting from a variation in blood pressure value is eliminated, so that obtained blood pressure values can be used without any processing and are unlikely to be affected by the personal property of the person to be examined, a condition thereof at the time of measurement, and the like. Thus, it is possible to obtain an accurate, universal, and objective vascular stiffness index. Note that the logarithm pulse pressure (ln(Ps/Pd)) is the logarithm of a ratio of the maximum blood pressure (systolic pressure: Ps) to the minimum blood pressure (diastolic pressure: Pd).

It is possible to provide a vascular stiffness evaluation apparatus in which the pulse wave velocity determining means calculates the pulse wave velocity (PWV) based on the following expression (2)
PWV=(L1−L2)/T1 Expression (2)
(where L1 denotes an arterial length between an aortic valve region and a popliteal region, L2 denotes an arterial length between the aortic valve region and a brachial artery beat region, and T1 denotes a time difference between a rising point of a brachial pulse wave and a rising point of a popliteal pulse wave).

The PWV is calculated using PWV=(L1−L2)/T1 (where L1 denotes an arterial length between an aortic valve region and a popliteal region, L2 denotes an arterial length between the aortic valve region and a brachial artery beat region, and T1 denotes a time difference between a rising point of a brachial pulse wave and a rising point of a popliteal pulse wave). Therefore, an accurate PWV value can be obtained. The arterial lengths are easily measured.

According to the present invention, the blood pressure detecting means including the strain sensor can be used. Conventional blood pressure measurement, for example, blood pressure measurement using an oscillometric method is likely to be affected by an external factor because an oscillation phenomenon of an internal cuff pressure is observed. The maximum pressure and the minimum pressure are determined by data processing based on software, so there is a problem in that an accurate blood pressure cannot be measured depending on software. On the other hand, when the strain sensor is used, a pulse pressure can be directly converted into an electrical signal to detect the blood pressure, so an accurate blood pressure value can be obtained.

When the pulse wave is detected by the strain sensor, a blood pressure at a time when a first negative notch which is not included in a preceding pulse waveform is caused can be set as the maximum blood pressure and a blood pressure at a time when the negative notch is lost can be set as the minimum blood pressure. When the maximum blood pressure and the minimum blood pressure are determined as described above, the maximum blood pressure and the minimum blood pressure can be easily and surely determined. The maximum blood pressure and the minimum blood pressure are accurate.

It is possible to provide a vascular stiffness evaluation apparatus in which the vascular stiffness index calculating means can calculate the vascular stiffness index based on a pulse wave velocity obtained by inputting existing data and a maximum blood pressure and a minimum blood pressure which are obtained by inputting existing data. When the vascular stiffness index is calculated based on the pulse wave velocity obtained by inputting existing data and the maximum blood pressure and the minimum blood pressure which are obtained by inputting existing data, a past state and a current state can be compared with each other using data related to PWV values and blood pressures, which are accumulated up to now.

According to the present invention, there is provided a vascular stiffness index calculating method including a step of calculating a pulse wave velocity based on pulse wave data and a step of calculating a vascular stiffness index based on blood pressure data and the pulse wave velocity, in which the pulse wave data is obtained before the blood pressure data is detected.

In the vascular stiffness index calculating method including the step of calculating the pulse wave velocity based on the pulse wave data and the step of calculating the vascular stiffness index based on the blood pressure data and the pulse wave velocity, the pulse wave data is obtained before the blood pressure data is detected, so the pulse wave data can be used as data which is not affected by compression during the blood pressure detection. Therefore, the pulse wave data is not affected by the vasoreflex or the vasospasm. When such data is used, a vascular stiffness index capable of accurately evaluating vascular stiffness is obtained. Assume that the pulse wave data widely includes data related to a pulse wave, such as a pulse wave, a pulse wave velocity, a pulse wave propagation time, or a pulse waveform and the blood pressure data widely includes data related to a blood pressure, such as a blood pressure, a maximum blood pressure, a minimum blood pressure, an average blood pressure, or a logarithm pulse pressure.

There is provided a vascular stiffness index calculating program that causes a computer to execute a process of calculating a vascular stiffness index which serves as an index of vascular stiffness of a living body using a predetermined calculation expression based on one of pulse wave data obtained by pulse wave detecting means including a strain sensor and existing pulse wave data obtained from an input terminal and one of blood pressure data obtained by blood pressure detecting means and existing blood pressure data obtained from the input terminal, and a process of outputting the calculated vascular stiffness index to display means.

The process of calculating the vascular stiffness index which serves as the index of vascular stiffness of the living body using the predetermined calculation expression based on one of the pulse wave data obtained by the pulse wave detecting means including the strain sensor and the existing pulse wave data obtained from the input terminal and one of the blood pressure data obtained by the blood pressure detecting means and the existing blood pressure data obtained from the input terminal, and the process of outputting the calculated vascular stiffness index to the display means are executed by the computer. Therefore, the computer can be operated as an arterial stiffness evaluation apparatus provided with the pulse wave detecting means including the strain sensor and the blood pressure detecting means, thereby making it possible to calculate the vascular stiffness index. Although raw data detected by the pulse wave detecting means and the blood pressure detecting means can be used as data related to the pulse wave and the blood pressure, the vascular stiffness index can be calculated based on existing data inputted from an input terminal of the computer. Thus, a latest state of the living body can be determined based on the raw data. In addition, a past state of the living body is determined based on data of the past and can be compared with a current state thereof.

It is possible to provide a vascular stiffness index calculating program which obtains the vascular stiffness index based on a value (stiffness) obtained by the following expression (1)
stiffness=ln(Ps/Pd)×k·PWV² Expression (1)
(where Ps denotes a maximum blood pressure, Pd denotes a minimum blood pressure, PWV denotes the pulse wave velocity, and k denotes a constant), and which can calculate the pulse wave velocity (PWV) based on the following expression (2)
PWV=(L1−L2)/T1 Expression (2)
(where L1 denotes an arterial length between an aortic valve region and a popliteal region, L2 denotes an arterial length between the aortic valve region and a brachial artery beat region, and T1 denotes a time difference between a rising point of a brachial pulse wave and a rising point of a popliteal pulse wave)

According to the present invention, an accurate and universal vascular stiffness index can be obtained without using, for example, calibration data set in advance and being affected by vasoreflex or vasospasm and the degree of vascular stiffness can be determined accurately and quickly.

The contents of the present invention should not be limited to the foregoing description. The objects, advantages, features, uses of the present invention will be made more apparent by the following description that will be given with reference to the accompanying drawings. It should also be understood that all suitable modifications are included in the scope of the present invention as long as they do not depart from the spirit thereof.

### BRIEF DESCRIPTION OF THE DRAWINGS

In the accompanying drawings:

FIGS. 1A and 1B show a brachial pulse wave detecting device, in which FIG. 1A is a sectional view taken along the SA-SA line shown in FIG. 1B and FIG. 1B is a plan view thereof;

FIG. 2 is a perspective view showing a state in which the brachial pulse wave detecting device is attached to an upper arm of a living body;

FIGS. 3A and 3B show a strain sensor, in which FIG. 3A is a front view showing a pressure transducer thereof and FIG. 3B is a plan view showing the strain sensor;

FIG. 4 is an amplifier block diagram showing circuits for processing data obtained by the strain sensor;

FIGS. 5A and 5B show a popliteal pulse wave detecting device, in which FIG. 5A is a sectional view taken along the SB-SB line shown in FIG. 5B and FIG. 5B is a plan view thereof;

FIG. 6 is a time chart showing pulse waveforms and other signals which are detected from the living body by the pulse wave detecting devices;

FIG. 7 is a time chart showing a waveform of a pulse wave detected by the strain sensor;

FIG. 8 shows a systematic blood pressure distribution characteristic;

FIG. 9 is a block diagram showing a vascular stiffness evaluation apparatus according to an embodiment of the present invention;

FIG. 10 is a block diagram showing a vascular stiffness evaluation apparatus according to another embodiment of the present invention;

FIG. 11 is a graph showing a correlation between stiffness and a PWV obtained based on a PWV original method;

FIG. 12 is a schematic view showing a conventional example of a pulse wave velocity measuring method;

FIG. 13 is a time chart showing pulse waveforms used to explain the conventional example of the pulse wave velocity measuring method;

FIG. 14 is a schematic view showing another conventional example of the pulse wave velocity measuring method;

FIG. 15 is a time chart showing pulse waveforms used to explain the other conventional example of the pulse wave velocity measuring method; and

FIG. 16 is a graph showing pulse wave velocity calibration curves.

### DETAILED DESCRIPTION OF THE PREFERRED EMBODIMENTS

Hereinafter, the present invention will be described in detail with reference to embodiments thereof. A vascular stiffness evaluation apparatus according to each of the embodiments includes a pulse wave detecting means, a pulse wave velocity determining means, a blood pressure detecting means, and a vascular stiffness index calculating means.

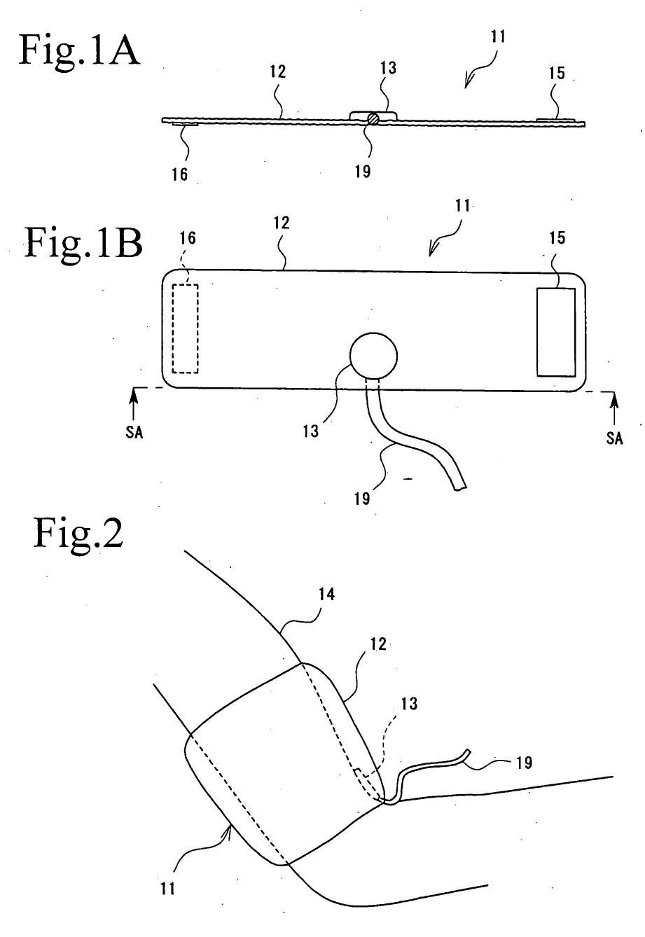

Pulse wave detecting means: the pulse wave detecting means includes a brachial pulse wave detecting device 11 for detecting a pulse wave from an upper arm of a living body and a popliteal pulse wave detecting device 21 for detecting a pulse wave from a popliteal region of the living body. Any device can detect the pulse wave from the living body and output, for example, a waveform of the detected pulse wave to a display. FIGS. 1A and 1B show an example of the brachial pulse wave detecting device 11. The brachial pulse wave detecting device 11 has a structure in which a strain sensor 13 is provided in a center portion of a band-shaped cuff 12 in a long-side direction and an end portion thereof in a short-side direction. Because the strain sensor 13 is provided on the cuff 12, as shown in FIG. 2, when an upper arm portion 14 is wound with the cuff 12 and the cuff 12 is held by, for example, Velcro (registered trademark) fasteners 15 and 16 stitched thereon, the strain sensor 13 can be pressed and held at a low pressure of the order of 10 mmHg in a state in which the strain sensor 13 is located immediately above a brachial artery beat region. The strain sensor 13 included in the brachial pulse wave detecting device 11 has high sensitivity, so the pulse wave can be accurately detected even when a slight variation in position occurs.

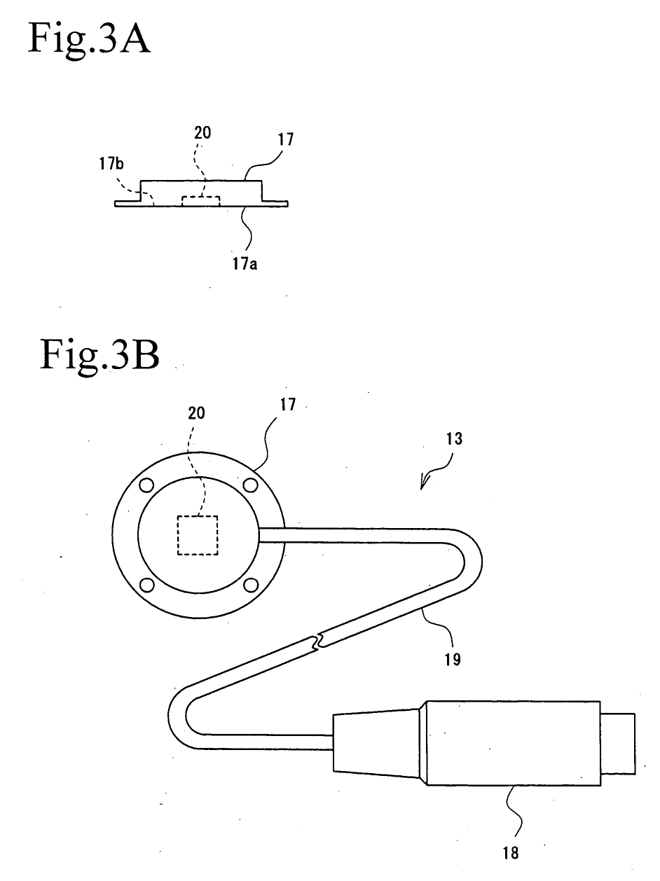

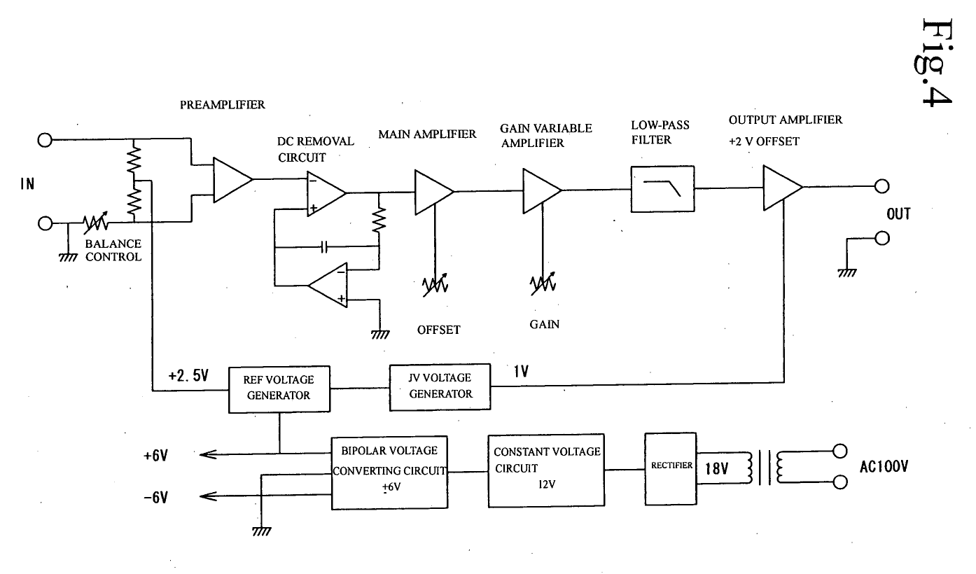

FIGS. 3A and 3B are external views showing the strain sensor 13. The strain sensor 13 includes a pressure transducer 17 and a semiconductor strain gauge 20. The pressure transducer 17 has a cylindrical or hat shape whose diameter is approximately 30 mm and thickness is approximately 5 mm to 20 mm. The pressure transducer 17 is coupled to a mini DIN plug (4P) 18 connected with an amplifier (not shown) through a cord 19. The semiconductor strain gauge 20 is provided on a rear surface 17b which is exposed in a surface 17a of the pressure transducer 17 and composed of a stainless steel plate or the like. When the strain sensor 13 receives a pressure (pulse pressure) from the living body, the semiconductor stain gauge 20 strains. The stain is converted into an electrical signal. The electrical signal is amplified by an amplifier, which is a part of the pulse wave detecting means (not shown) (FIG. 4).

It is only necessary that the cuff 12 used for the brachial pulse wave detecting device 11 does not press a pulse wave detection region to stop a blood flow but holds the strain sensor 13 so as not to be displaced during the pulse wave detection as in the case of a cuff used for a blood pressure meter which is based on an oscillometric method. Note that it is preferable that the brachial pulse wave detecting device 11 also act as the blood pressure detecting means, so the cuff 12 capable of applying a pressure to a measurement region to stop the blood flow can be used.

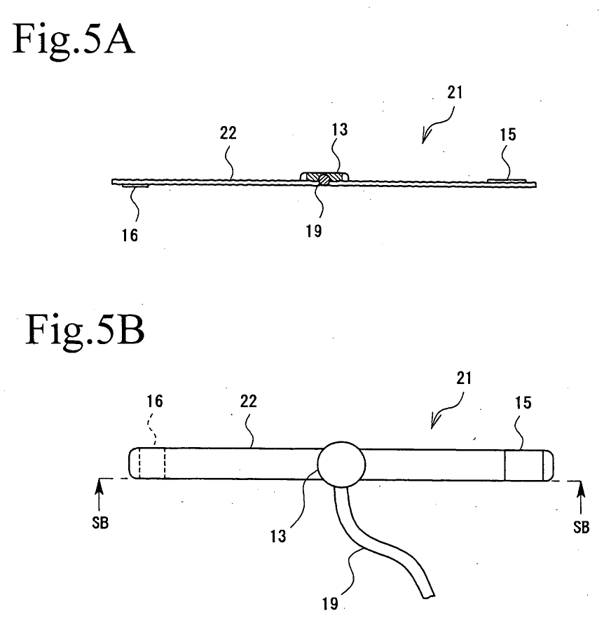

The popliteal pulse wave detecting device 21 may be identical to the brachial pulse wave detecting device 11. However, when it is unnecessary to measure a blood pressure of the popliteal region or when a femoral region is wound with a cuff to put mental and physical burdens on a person to be examined, a band-shaped binding tool 22 to which the strain sensor 13 is attached as shown in FIGS. 5A and 5B, such as a Velcro (registered trademark) band having a short width is preferably used instead of the cuff. In addition to the Velcro (registered trademark) band, a binding tool made of cloth or a binding tool in which rubber is taken in a cloth to have a stretch property can be used as the band-shaped binding tool 22. The Velcro (registered trademark) fasteners 15 and 16 are preferably used for a binding portion. When the stretch property is large, a ring-shaped fastening tool without the binding portion may be used. Note that it is necessary to prevent the popliteal region from extremely fastening at a pressure which exceeds 30 mmHg.

A carotid artery pulse wave sensor for detecting the carotid artery pulse wave caused from the carotid artery, a femoral artery pulse wave sensor for detecting the femoral artery pulse wave caused from the femoral artery, which is in contact with an inguinal region, and a heart sound sensor for detecting a heart sound caused from a heart, which is placed immediately above the heart, each can be included as the pulse wave detecting means. An electrocardiogram lead device including a plurality of electrodes attached to both wrists to obtain electrocardiogram waveforms may be included.

When the popliteal region and the brachial region are set as the pulse wave detection regions and the strain sensor 13 is used as the pulse wave detecting means, it is possible to solve the problem caused in the conventional CAVI apparatus. That is, in the conventional CAVI apparatus, approximately 20% of all measurement cases include an abnormal result. However, when the apparatus according to the present invention (hereinafter referred to as a “stiffness apparatus”) is used, an abnormal result does not occur. This reason is as follows. In the conventional CAVI apparatus, limb arteries other than the aorta, particularly, ankles lower than knees and wrists anterior to elbows are wound with cuffs to measure pulse wave velocities of blood vessels of various regions of the body. Therefore, vasoreflex or vasospasm occurs, so that there is a problem in that the pulse wave velocity significantly increases. In the present invention, this problem is solved.

The limb arteries such as upper limb arteries and lower limb arteries have different properties and different functions in cyclical physiology or vascular biophysics from central arteries such as a carotid artery, thoracic and abdominal aortas, and a common iliac artery. However, a problem in the CAVI apparatus is that the limb arteries are lumped together as the artery and easily assumed based on the same standard. According to understanding though the current improvement, it is necessary to carefully assume the limb arteries such as the upper limb arteries and the lower limb arteries.

Pulse wave velocity determining means: the pulse wave velocity determining means determines the pulse wave velocity by dividing an arterial length between two points of the living body at which pulse waves are detected by a pulse wave transmission time. A specific pulse wave velocity determining method will be described below.

The arterial length between the two points of the living body at which the pulse waves are detected is a distance between the brachial region (more properly, brachial artery beat region) and the popliteal region. Therefore, it is necessary to obtain the arterial length between the brachial artery beat region and the popliteal region. The pulse waves propagate on the same path between the heart and an aortic arch, so a distance obtained by subtracting an arterial length L2 between the aortic valve region and the brachial region from an arterial length L1 between the aortic valve region and the popliteal region is substantially set.

Although an estimated value calculated from a body height can be used as the distance between the pulse wave detection regions, there are great differences between individuals, so that an error may be large. Therefore, a measured value is preferably used to obtain a more accurate evaluation. However, it is difficult to measure the arterial length L1 between the aortic valve region and the popliteal region. It has been demonstrated that 1.3 times a measured straight distance between a second intercostal sternal edge and a contralateral femoral artery beat region may be used as the arterial length between the aortic valve region and the inguinal region. Thus, the arterial length L1 can be obtained by adding a measured distance between the inguinal region and the popliteal region to the distance (1.3 times the measured straight distance). A femoral artery length between the inguinal region and the popliteal region is straight and can be easily measured. The arterial length L2 between the aortic valve region and the brachial region can be also measured.

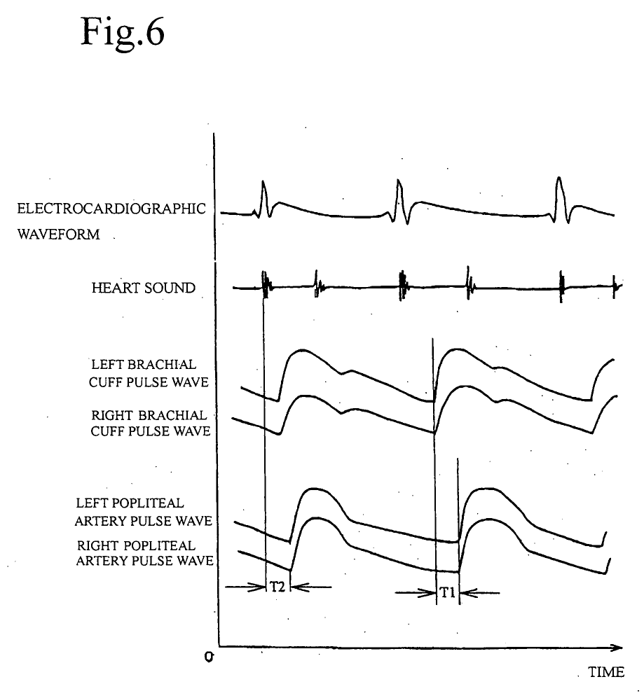

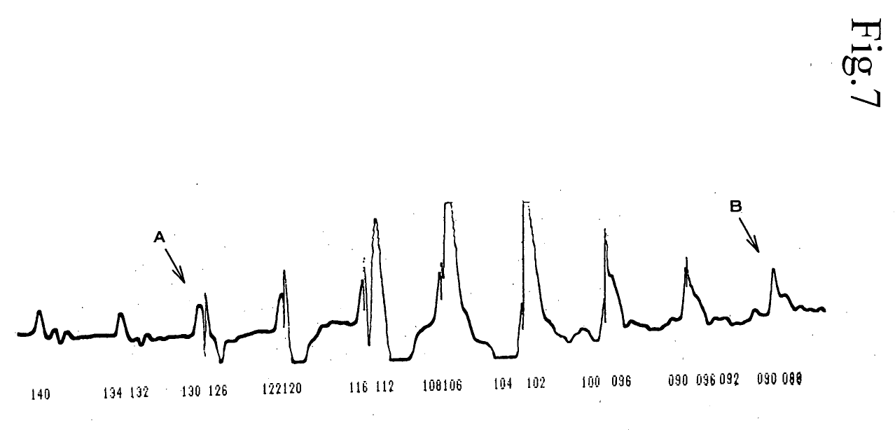

On the other hand, a pulse wave propagation time can be obtained based on a pulse waveform. FIG. 6 is a time chart in which pulse waves, an electrocardiogram waveform, and a heart sound, which are detected by various pulse wave detecting means, are shown on a common time base. A pulse wave propagation time between the brachial region and the popliteal region is a difference between a pulse wave propagation time between the aortic valve region and the popliteal region and a pulse wave propagation time between the aortic valve region and the brachial region. Therefore, a time difference between a rising point of a brachial artery pulse wave and a rising point of a popliteal artery pulse wave may be obtained. This time difference is set as a time T1.

The pulse wave velocity is calculated by (L1−L2)/T1 based on the distance (L1−L2) between the pulse wave detection regions and the pulse wave propagation time (T1) which are obtained as described above.

Instead of using the brachial pulse wave detecting device 11, the heart sound sensor may be used. A pulse wave propagation time T2 between the aortic valve region and the popliteal region is measured based on, for example, a heart sound obtained by the heart sound sensor for detecting the heart sound. The arterial length L1 is divided by the pulse wave propagation time T2, so that the pulse wave velocity can be obtained.

Blood pressure detecting means: blood pressure detecting means detects blood pressures, particularly, the maximum blood pressure of the living body and the minimum blood pressure thereof and includes the strain sensor 13. Therefore, the brachial pulse wave detecting device 11 can be used as the blood pressure detecting means. However, when the maximum blood pressure and the minimum blood pressure are to be detected, a measurement region is compressed to temporarily close the artery and then a pressure to the measurement region is gradually reduced to check a change in pulse wave. Therefore, a cuff which can merely wound around an arm or a foot is insufficient, so that it is necessary to use a cuff serving as a compression band capable of compressing the arm or the foot to reduce the blood flow thereof. Such a cuff may be a cuff used for a blood pressure meter based on the oscillometric method.

The maximum blood pressure and the minimum blood pressure are obtained based on a change of a pulse waveform which is detected by the strain sensor 13 after the closed artery is released. FIG. 7 shows a pulse waveform produced during a change of a compression pressure of the cuff wound around the brachial region. As is apparent from FIG. 7, the pulse waveform changes during a reduction in cuff pressure. In the pulse waveform, the maximum blood pressure is set as a blood pressure at a time when a negative notch which is not included in a preceding pulse waveform is caused as a forward waveform component (“A” shown in FIG. 7). In addition, the minimum blood pressure is set as a blood pressure at a time when the negative notch is lost (“B” shown in FIG. 7) As described above, the maximum blood pressure and the minimum blood pressure are identified corresponding to the appearance and disappearance of the negative notch of the pulse waveform, with the result that the maximum blood pressure and the minimum blood pressure can be easily determined. It is known that the maximum blood pressure and the minimum blood pressure which are obtained using this method are equal to a maximum blood pressure and a minimum blood pressure which are measured using an invasive method of inserting a catheter into a radial artery and thus these blood pressures are accurate values.

Various types of blood pressure meters are based on the oscillometric method, a Korotkoff sound detection method, a tonometry method, or the like. However, an accurate maximum blood pressure and an accurate minimum blood pressure cannot be measured using any of the methods. For example, in the measurement of the maximum blood pressure and the accurate minimum blood pressure using the oscillometric method, a pulse pressure is not directly detected but obtained from an oscillation phenomenon of an internal cuff pressure. Therefore, there is such a defect that a noise caused by an external factor is likely to be interfered. The occurrence of oscillation is complex, so it is likely to be significantly affected by software for performing computer processing on an oscillation pattern. When the tonometry method is used, there are the following defects. That is, it is necessary to uniformly compress a superficial artery such as the radial artery from a body surface. In addition, it is necessary that several tens of small pressure sensors are provided and an adequate output is selected from a plurality of outputs obtained from the pressure sensors. However, the blood pressure detecting means using the strain sensor 13 has no defects as described above and thus is a low-cost device capable of accurately obtaining the maximum blood pressure and the minimum blood pressure.

A result obtained by blood pressure detection using the blood pressure detecting means is preferably prevented from being reflected in the pulse wave velocity even if vasoreflex or vasospasm occurs. Therefore, it is preferable to detect the pulse wave before the detection of the maximum blood pressure and the minimum blood pressure.

The blood pressure detection region is preferably to set to any region between at least two pulse wave detection subject regions for determining the pulse wave velocity. This is because the pulse wave velocity is affected by the blood pressure, and the blood pressure affecting the obtained pulse wave velocity is actually reflected.

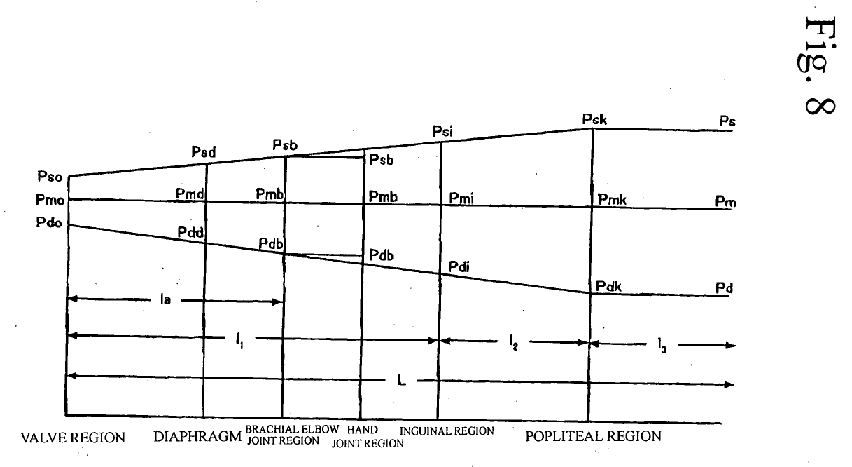

FIG. 8 shows a systematic body blood pressure distribution characteristic, that is, a relationship between each region of the living body and a pressure. A maximum blood pressure and a minimum blood pressure of each of regions including the aortic valve region to a hand joint or a foot joint depend on individuals. While a blood pressure detection region is shifted to a peripheral region, the maximum blood pressure increases and the minimum blood pressure slightly decreases. Therefore, the pulse pressure increases and an average blood pressure is substantially constant In addition, a maximum blood pressure and a minimum blood pressure of a brachial elbow joint region are substantially equal to those of the hand joint and a maximum blood pressure and a minimum blood pressure of a popliteal region are substantially equal to those of the foot joint. A maximum blood pressure of a desirable region and a minimum blood pressure thereof can be obtained from the maximum blood pressure of the pulse wave detection region and the minimum blood pressure thereof based on the systematic body blood pressure distribution characteristic. It is preferable to adjust a blood pressure value of the measurement region to, for example, a blood pressure value obtained at a center point of a section in which the pulse wave velocity is measured, using the systematic body blood pressure distribution characteristic, to calculate the vascular stiffness index.

Vascular stiffness index calculating means: the vascular stiffness index calculating means calculates the vascular stiffness index using the following expression (1) based on pulse wave data including the pulse waveform, the pulse wave velocity, or a blood vessel length and blood pressure data including the maximum blood pressure and minimum blood pressure, the average blood pressure, or a logarithm pulse pressure (ln(Ps/Pd)), particularly, the pulse wave velocity obtained by the pulse wave velocity determining means and the maximum and minimum blood pressures obtained by the blood pressure detecting means.
stiffness=ln(Ps/Pd)×k·PWV² Expression (1)
(where Ps denotes a maximum blood pressure, Pd denotes a minimum blood pressure, PWV denotes a pulse wave velocity, and k denotes a constant).

In the expression (1), there is the following relationship between a value related to the logarithm pulse and a value related to the pulse wave velocity. That is, when one value increases, the other value conversely decreases. On the other hand, when one value decreases, the other value conversely increases. Therefore, the product of the value related to the logarithm pulse pressure and the value related to the pulse wave velocity is a specific value reflecting the vascular stiffness of the living body and becomes the vascular stiffness index accurately reflecting the vascular stiffness of the living body.

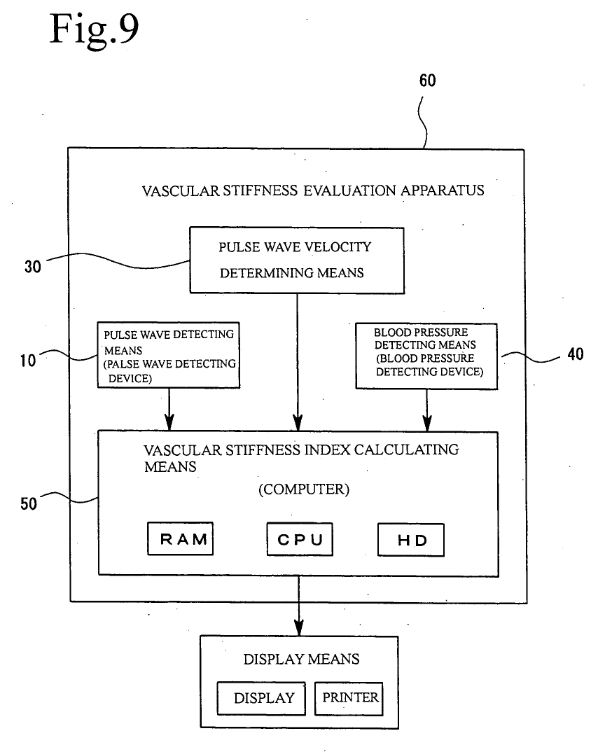

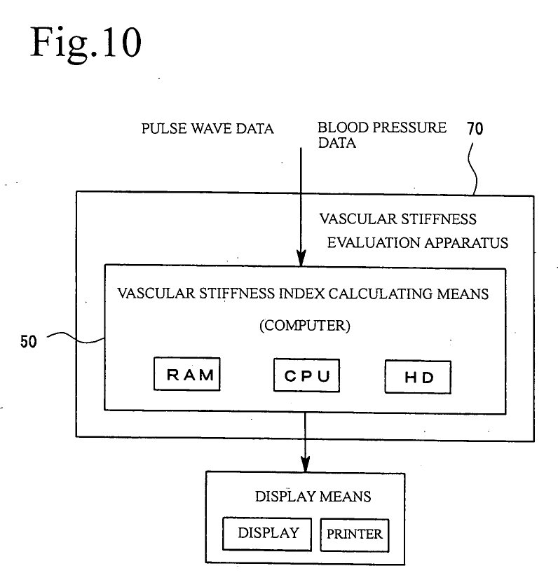

FIGS. 9 and 10 are block diagrams showing vascular stiffness evaluation apparatuses 60 and 70 according to different embodiments of the present invention. The vascular stiffness evaluation apparatus 60 shown in FIG. 9 includes a pulse wave detecting means 10, a pulse wave velocity determining means 30, a blood pressure detecting means 40, and a vascular stiffness index calculating means 50. The vascular stiffness index calculating means 50 includes a computer and a computer program for the computer. The computer has a central processing unit (CPU), a random access memory (RAM), and a hard disc (HD) drive. For example, assume that a vascular stiffness index calculating program recorded in an external recording medium such as a CD-ROM is read out on the RAM and executed by the CPU. In this case, the pulse wave data obtained by the pulse wave detecting means 10, the blood pressure data including the maximum blood pressure and the minimum blood pressure, which is obtained by the blood pressure detecting means 40, and data including a predetermined blood vessel length, which is inputted from an outside, are incorporated in a predetermined calculation expression to calculate the vascular stiffness index. The calculated vascular stiffness index is displayed on a display or printed on a printer together with a patient name, data of the past, and the like.

The vascular stiffness evaluation apparatus 70 shown in FIG. 10 has substantially the same function as that of the vascular stiffness evaluation apparatus 60 shown in FIG. 9. However, data obtained by the pulse wave detecting means 10 and the blood pressure detecting means 40 are not used. Instead of this, existing pulse wave data and existing blood pressure data are inputted to a computer which is the vascular stiffness index calculating means 50. Therefore, the vascular stiffness index calculating means 50 can use data of the past.

The pulse wave data and the blood pressure data which are used in the vascular stiffness index calculating means 50 may be not the pulse wave velocity and the blood pressure value but pulse waveform data or the like. In addition, it is possible to use existing pulse wave velocities measured using various PWV methods, which are accumulated up to now. Each of the apparatuses according to the present invention is compatible with data obtained using the PWV methods. Therefore, it is possible to use fundamental data, epidemiological data, clinical data, and drug efficacy data, which are obtained based on the PWV methods substantially over the past 40 years.

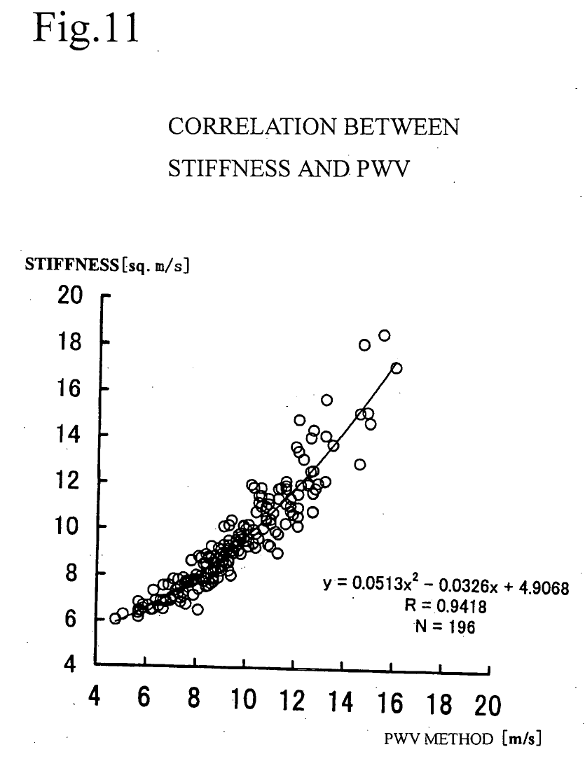

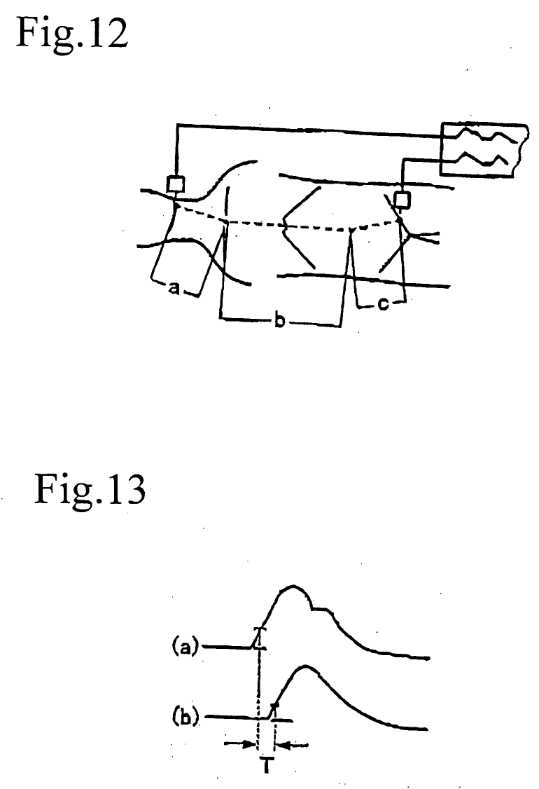

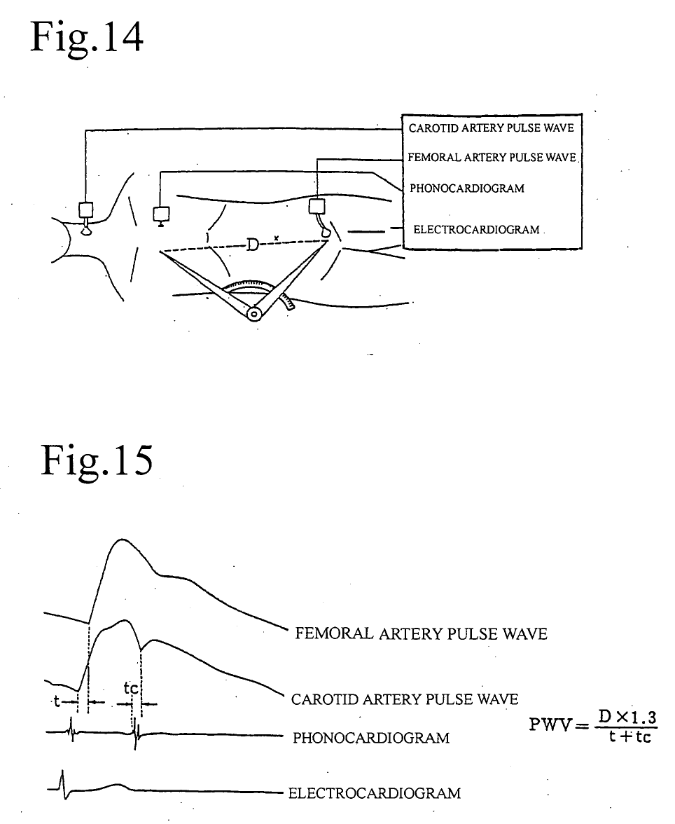

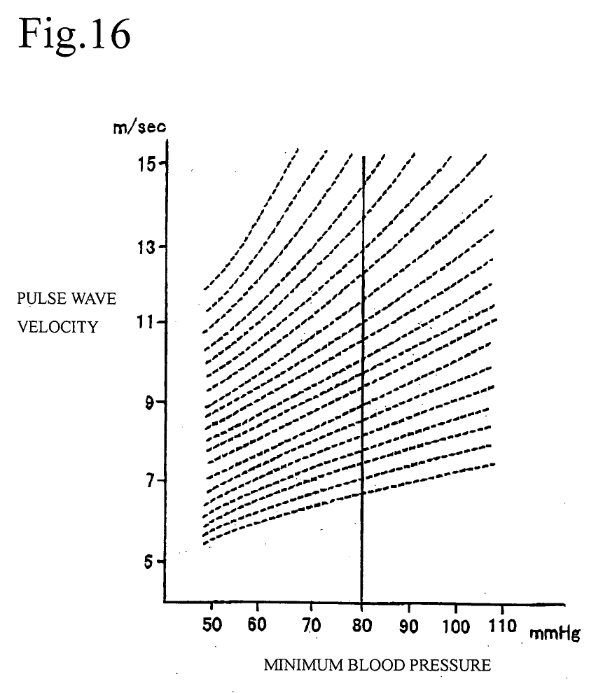

FIG. 11 shows a correlation between a stiffness value and a PWV value measured using a PWV original method. Those values between the aortic valve region and the femoral artery beat region are calculated and compared with each other. The number of subject cases is 196. The correlation is obtained with a quadratic function relationship and a high correlation coefficient r of 0.9418 is obtained. Therefore, when a quadratic functional expression is applied, each data related to the PWV original method can be converted into a stiffness value.

The stiffness value which is the vascular stiffness index calculated as described above is outputted as an index indicating vascular stiffness to a display or a printer together with the data of the past. Therefore, the stiffness value can be used for the diagnosis of arteriosclerosis.

What is claimed is:

1. A vascular stiffness evaluation apparatus, comprising:
pulse wave detecting means for detecting pulse waves, which comprises a strain sensor;
pulse wave velocity determining means for determining a pulse wave velocity based on the pulse waves;
blood pressure detecting means for detecting blood pressures; and
vascular stiffness index calculating means for calculating a vascular stiffness index which serves as an index of vascular stiffness of a living body based on the pulse wave velocity and the blood pressures.

2. A vascular stiffness evaluation apparatus according to claim 1, wherein
the vascular stiffness index is based on a value obtained by an expression
stiffness=ln(Ps/Pd)×k·PWV²
(where Ps denotes a maximum blood pressure, Pd denotes a minimum blood pressure, PWV denotes the pulse wave velocity, and k denotes a constant).

3. A vascular stiffness evaluation apparatus according to claim 1, wherein
the pulse wave detecting means comprises brachial pulse wave detecting means for detecting a brachial pulse wave of the living body and popliteal pulse wave detecting means for detecting a popliteal pulse wave of the living body.

4. A vascular stiffness evaluation apparatus according to claim 1, wherein
the pulse wave velocity determining means calculates the pulse wave velocity (PWV) based on an expression
PWV=(L1−L2)/T1
(where L1 denotes an arterial length between an aortic valve region and a popliteal region, L2 denotes an arterial length between the aortic valve region and a brachial artery beat region, and T1 denotes a time difference between a rising point of a brachial pulse wave and a rising point of a popliteal pulse wave).

5. A vascular stiffness evaluation apparatus according to claim 1, wherein
the vascular stiffness index calculating means can calculate the vascular stiffness index based on a pulse wave velocity obtained by inputting existing data and based on a maximum blood pressure and a minimum blood pressure which are obtained by inputting existing data.

6. A vascular stiffness evaluation apparatus according to claim 1, wherein
the strain sensor of the pulse wave detecting means comprises a pressure transducer including a semiconductor strain gauge.

7. A vascular stiffness evaluation apparatus according to claim 1, wherein
the blood pressure detecting means comprises a strain sensor.

8. A vascular stiffness evaluation apparatus according to claim 7, wherein
the strain sensor of the blood pressure detecting means comprises a pressure transducer including a semiconductor strain gauge.

9. A vascular stiffness evaluation apparatus according to claim 7, wherein
a pressure at a time when a first negative notch which is not included in a preceding pulse waveform is caused in a pulse waveform obtained by the strain sensor of the pulse wave detecting means is set as a maximum blood pressure, and a pressure at a time when the first negative notch is lost is set as a minimum blood pressure.

10. An arteriosclerosis diagnosis method, comprising:
determining a pulse wave velocity based on pulse waves detected by pulse wave detecting means which comprises a strain sensor; and
calculating a vascular stiffness index which serves as an index of vascular stiffness of a living body based on blood pressures obtained by blood pressure detecting means and the determined pulse wave velocity to determine a degree of arteriosclerosis.

11. An arteriosclerosis diagnosis method according to claim 10, wherein
the vascular stiffness index is based on a value obtained by an expression
stiffness=ln(Ps/Pd)×k·PWV²
(where Ps denotes a maximum blood pressure, Pd denotes a minimum blood pressure, PWV denotes the pulse wave velocity, and k denotes a constant).

12. An arteriosclerosis diagnosis method according to claim 10, wherein
the pulse waves comprises pulse waves obtained from an upper arm of the living body and a popliteal region thereof.

13. An arteriosclerosis diagnosis method according to claim 10, wherein
the pulse waves are detected by the pulse wave detecting means before the blood pressures are detected by the blood pressure detecting means.

14. An arteriosclerosis diagnosis method according to claim 10, wherein
the strain sensor of the pulse wave detecting means comprises a pressure transducer including a semiconductor strain gauge.

15. An arteriosclerosis diagnosis method according to claim 10, wherein
the blood pressure detecting means comprises a strain sensor.

16. An arteriosclerosis diagnosis method according to claim 15, wherein
the strain sensor of the blood pressure detecting means comprises a pressure transducer including a semiconductor strain gauge.

17. An arteriosclerosis diagnosis method according to claim 15, wherein
a pressure at a time when a first negative notch which is not included in a preceding pulse waveform is caused in a pulse waveform obtained by the strain sensor of the pulse wave detecting means is set as a maximum blood pressure, and a pressure at a time when the first negative notch is lost is set as a minimum blood pressure.

18. A vascular stiffness index calculating program for causing a computer to execute the processings of:
calculating a vascular stiffness index which serves as an index of vascular stiffness of a living body using a predetermined calculation expression based on one of pulse wave data obtained by pulse wave detecting means including a strain sensor and existing pulse wave data obtained from an input terminal and one of blood pressure data obtained by blood pressure detecting means and existing blood pressure data obtained from the input terminal; and
outputting the calculated vascular stiffness index to display means.

19. A vascular stiffness index calculating program according to claim 18, wherein
the vascular stiffness index is based on a value obtained by an expression
stiffness=ln(Ps/Pd)×k·PWV²
(where Ps denotes a maximum blood pressure, Pd denotes a minimum blood pressure, PWV denotes the pulse wave velocity, and k denotes a constant).

20. A vascular stiffness index calculating program according to claim 18, wherein
the pulse wave velocity determining means calculates the pulse wave velocity (PWV) based on an expression
PWV=(L1−L2)/T1
(where L1 denotes an arterial length between an aortic valve region and a popliteal region, L2 denotes an arterial length between the aortic valve region and a brachial artery beat region, and T1 denotes a time difference between a rising point of a brachial pulse wave and a rising point of a popliteal pulse wave).
